(19) United States
(12) Patent Application Publication
(10) Pub. No.: US 2014/0296796 A1
(43) Pub. Date: Oct. 2, 2014

(54) PLASTIC MICRONEEDLE STRIP

(71) Applicant: Chee Yen Lim, Singapore (SG)

(72) Inventor: Chee Yen Lim, Singapore (SG)

(21) Appl. No.: 14/351,899

(22) PCT Filed: Nov. 2, 2011
(86) PCT No.: PCT/SG2011/000392

§ 371 Date: Apr. 15, 2014

(51) Int. Cl. A61M 37/00; B29C 33/38

(52) U.S. Cl. CPC A61M 37/0015; B29C 33/3842; A61M 2037/0023; A61M 2037/0046; A61M 2037/0053
USPC 604/272; 29/428

(57) ABSTRACT

The present invention relates to plastic microneedle strips (10) that are used in transdermal drug delivery for increasing the drug delivery rate through the skin These microneedle strips (10) are mass produced singly or in a cluster (100) via precision injection moulding process, and may be assembled to form microneedle arrays subsequently. The individual microneedle strips can further broken into individual microneedles for other medical applications. The making of the mould (200) for precision injection moulding process is also provided.

### FIELD OF THE INVENTION

The present invention relates to microneedles for transdermal drug delivery, particularly, it relates to making of cost-efficient plastic microneedle strips which can be assembled into array forms for drug delivery purposes.

### BACKGROUND OF THE INVENTION

Drug is normally administered or brought to contact with a body via topical, enteral (oral) and parenteral (injection) means. In topical administration, the applied drug is supposed to take effect locally, while in enteral and parenteral administration, the drug effect is systemic (entire body). Transdermal drug delivery is a relatively new form of drug administration targeting a systemic delivery by making drugs available on the skin. This is different than topical method as it normally targets local delivery. This is the first obstacle as the efficacy of the drug is not guaranteed even the drug can be successfully delivered to the body.

The second obstacle in transdermal delivery is to overcome the outermost layer of the skin, called stratum corneum, which is made up by dead cells that are pushed to the outermost of the body. Stratum corneum forms a formidable layer (20 microns on average) to isolate and protect the body. Because of this formidable layer, only a few small-molecule drugs can be administered via transdermal route. Over two decades, transdermal delivery is complemented by microneedles to overcome stratum corneum to allow faster delivery rate and larger-molecule drugs to be delivered. Since microneedles physically breach or perforate the skin to make way for the drug, the effectiveness is excellent and consistent.

After examining the first two obstacles, there is the last obstacle that is in the way of transdermal delivery, i.e. the cost of microneedles, which includes the initial capital investment and subsequent operational expenditure. Most microneedles developed in the lab lack the capability to scale up with acceptable cost efficiency. The Microneedle technology for transdermal drug delivery has been around for two decades and there is yet any commercial product on the market to date. One major hindrance for commercialization is the production cost and mass manufacturability, which the present invention seeks to address.

### SUMMARY OF THE INVENTION

In one aspect, the present invention relates to plastic microneedle strips that can be used in transdermal drug delivery applications. Individual microneedles are connected in a row by a strip that is injection moulded. Each strip can be moulded singly or in a cluster within a mould cavity. The height of the microneedles can range from 300 microns to 3000 microns, and 400 microns to 600 microns for most applications. The microneedles on each strip may have a uniform height or may have varying height such that the middle ones have greater height.

### SUMMARY OF THE INVENTION

In one aspect, the present invention relates to plastic microneedle strips that can be used in transdermal drug delivery applications. Individual microneedles are connected in a row by a strip that is injection moulded. Each strip can be moulded singly or in a cluster within a mould cavity. The height of the microneedles can range from 300 microns to 3000 microns, and 400 microns to 600 microns for most applications. The microneedles on each strip may have a uniform height or may have varying height such that the middle ones have greater height.

In another aspect, the present invention relates to the fabrication of such plastic microneedle strips, which involves a pair of mould inserts, one half having a surface that is substantially flat and smooth, and the other half having a surface engraved with the negative microneedle patterns, wherein both halves are to be coupled with the flat and smooth surface and the engraved surface in contact with each other. A plurality of such mould inserts can be employed in one mould cavity such that several strips can be produced in one process cycle.

### BRIEF DESCRIPTION OF THE DRAWINGS

FIG. 1 is a perspective view of plastic microneedle strips.

FIG. 2 is a close-up view of the plastic microneedles.

FIG. 3 is a close-up view of plastic microneedles with off-centred and centred peaks.

FIG. 4 is a close-up view of a triangular plastic microneedle.

FIG. 5 is a close-up view of a bevelled conical plastic microneedle.

FIG. 6 is a perspective view of a cluster of plastic microneedle strips

FIG. 7 is a perspective view of a set of mould inserts for moulding plastic microneedle strips.

### DETAILED DESCRIPTION OF THE INVENTION

For the purpose of illustrating the principles of the present invention, reference will now be drawn to the embodiments illustrated herein and specific language will be used to describe the same. It should be understood that no limitation of the scope of the present invention by these embodiments and language is intended. Any alterations and further modifications and applications of the principles of the present invention by a person skilled in the art shall fall in the scope of the present invention.

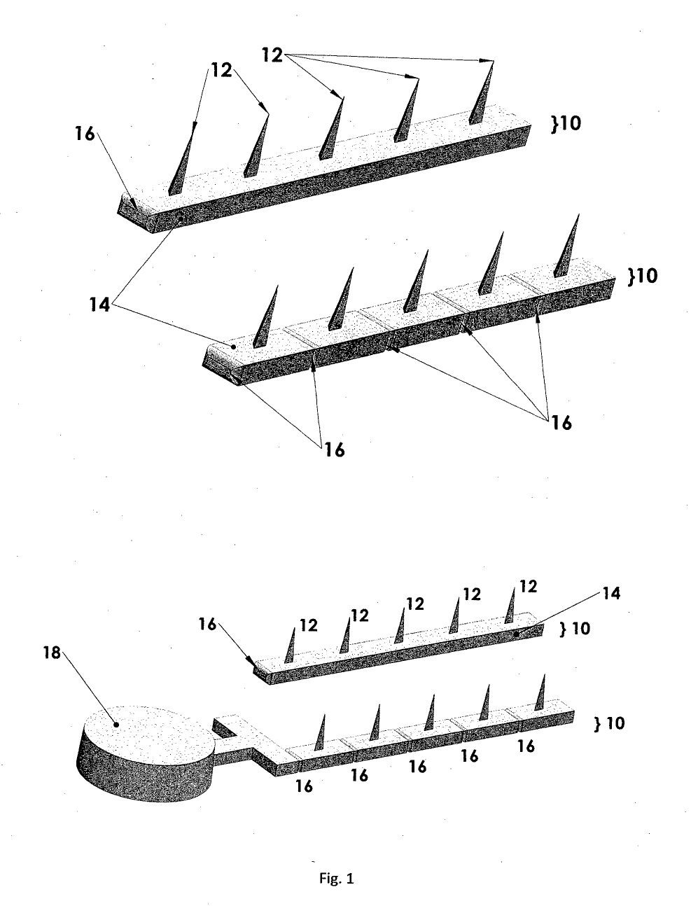

FIG. 1 refers to two plastic microneedle strips 10 comprising a plurality of microneedles 12 and a thin structure 14 which connects all the microneedles 12. Within the thin structure 14, there is at least a breaking notch 16 with abrupt reduction in cross-sectional area to facilitate breaking of the strip 14 from the rest of the moulded part 18 (e.g. moulded runner and gate). The breaking notch 16 can also be disposed along the thin structure 14 if it is intended to be broken into individual microneedles or shorter strips. The microneedles have a height ranging from 300 microns to 3000 microns; or in practice from 400 microns to 1000 microns for transdermal drug delivery applications.

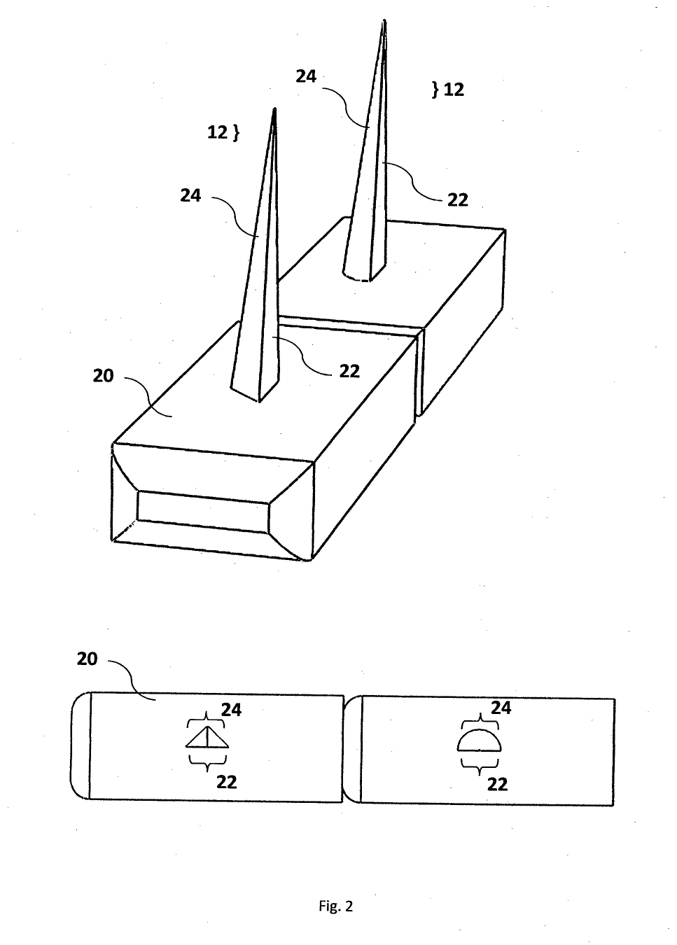

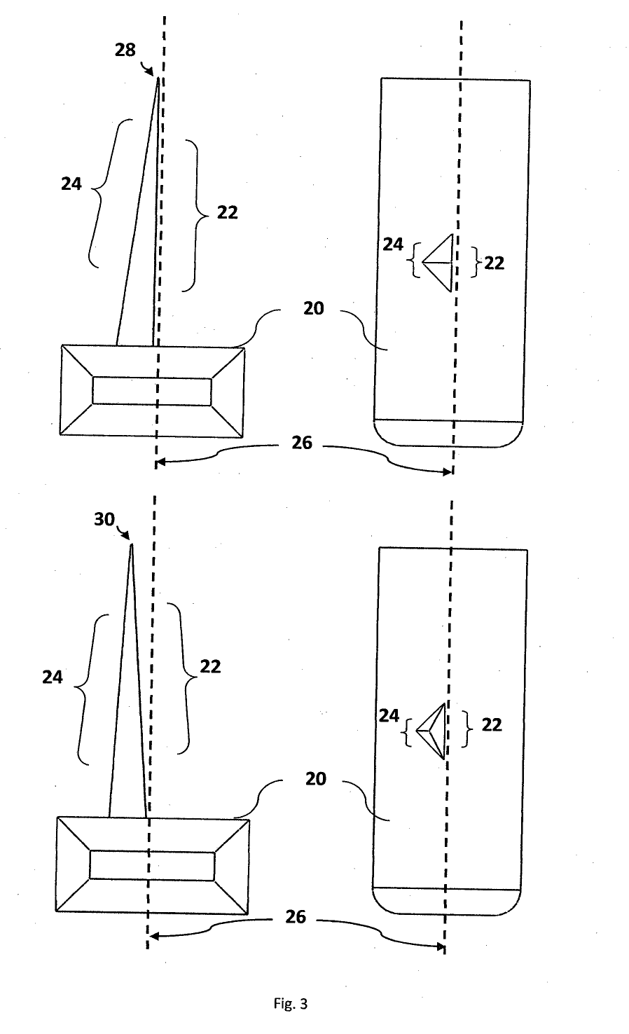

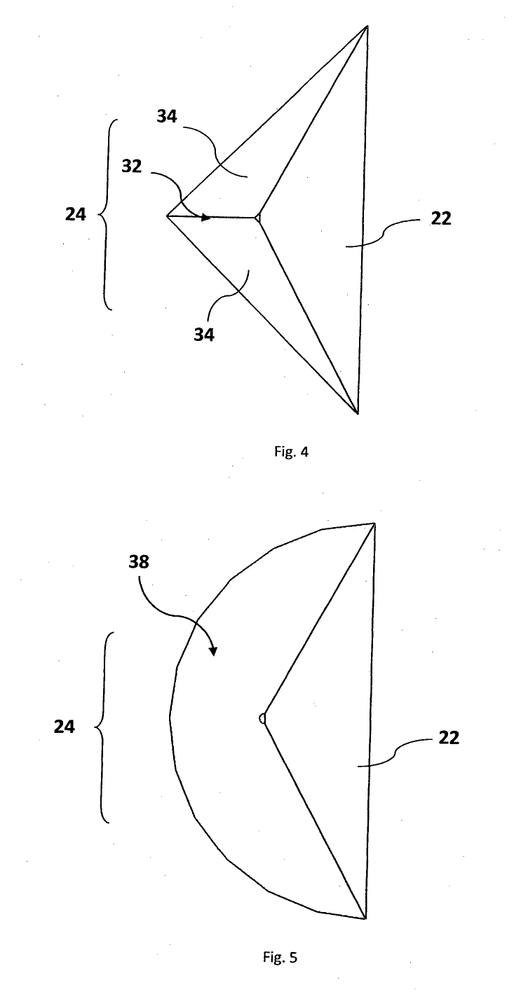

FIG. 2 shows the possible geometry of the microneedles 12 on a base surface 20. The LO microneedles 12 have in general a tapered triangular or halved-conical shape which comprises one substantially flat surface 22 and a tapered curved surface 24. As shown in FIG. 3, the flat surface 22 may be normal to the base surface 20 or slightly tilted such that the tip can be located at the centre of the microneedles in the top view. The dashed line 26 clearly differentiates the normal and tilted flat surface 22 which results into off-centred peak 28 and centred peak 30. As shown in FIG. 4, the tapered curved surface 24 has an edge 32 which divides the curved surface into two facets 34 at an acute angle, which together with the flat surface 22 forms a full triangle. Alternatively, as in FIG. 5, the tapered curved surface 24 can be a conical surface 38, which together with the flat surface 22 forms a bevelled cone.

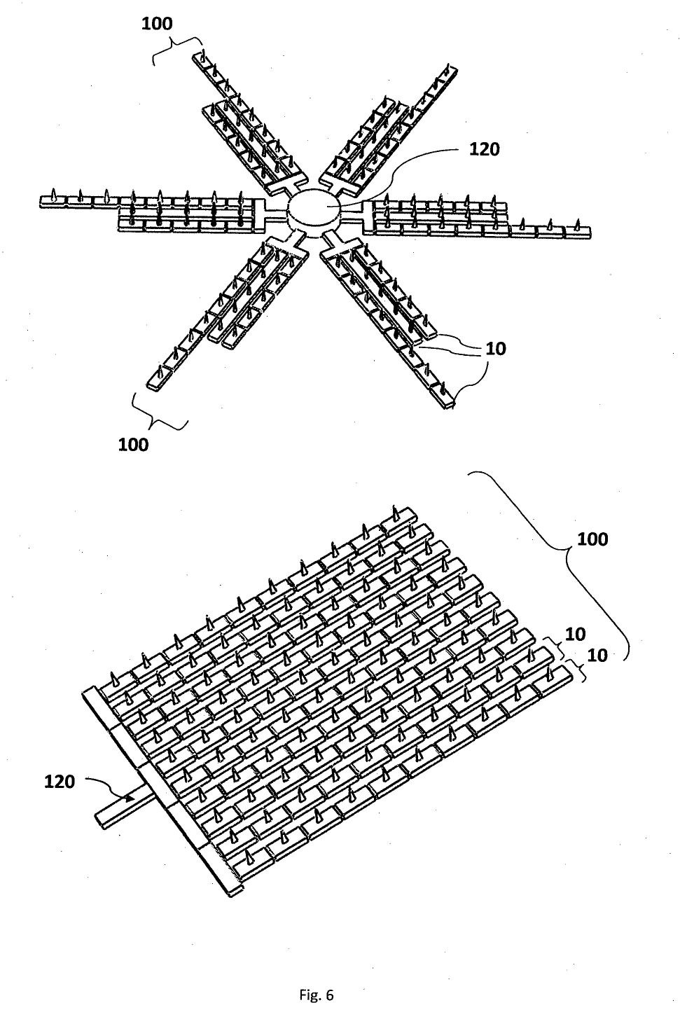

For cost efficiency reason, the microneedle strips 10 in FIG. 1 are normally injection moulded in clusters, i.e. there is a plurality of microneedles strips 10 in one mould cavity. FIG. 6 shows a cluster of microneedle strips 100 which consists of a plurality of microneedle strips 10. The individual microneedle strips 10 within a cluster 100 in practice may vary in the strip geometry (e.g. size, number of breaking notch 16 along a strip) and the needle's size, shape, height and spacing. There is one breaking notch 16 at the proximal end of the microneedle strips 10 which is linked to the main stem 120.

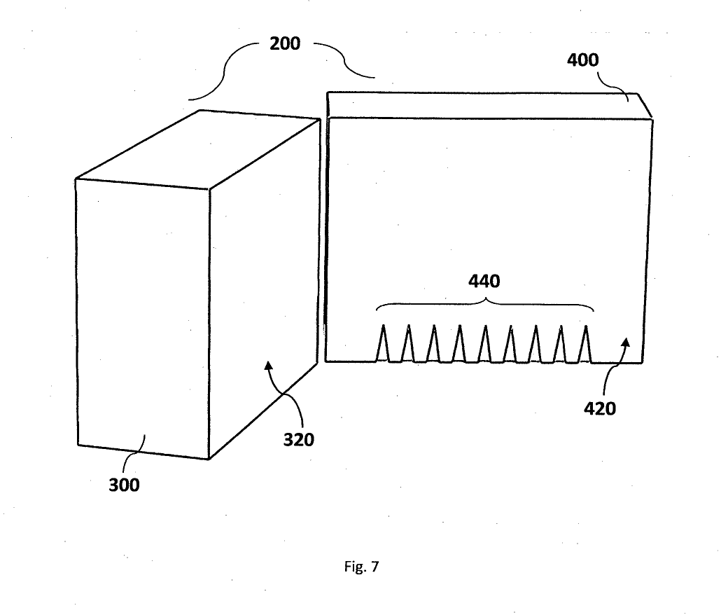

Now the production method, in particular, the making of the mould inserts, is described. FIG. 7 shows a set of mould inserts 200, comprising one blank insert 300 with a coupling surface 320 that is substantially flat and smooth, and one patterned insert 400 with a coupling surface 420 that is engraved with negative microneedle patterns 440. The methods for engraving the microneedle patterns include ultra-precision machining such as profile grinding, Electro-Discharge-Machining (EDM) wire cutting, and other unconventional methods. The coupling surfaces 320 and 420 are coupled together to define the outline of the negative microneedle patterns 440. The further steps in using this pair of mould inserts 200 is well understood by the persons skilled in the arts and will not be further elaborated. In normal circumstances, this method will produce microneedles with off-centred peaks 28 as shown in FIG. 3. Nonetheless, sometimes symmetrical shapes are desired, and this can be achieved by duly tilting the mould inserts 200 according to FIG. 3, which can be easily performed by the persons skilled in the art.

Some preclinical evaluations were carried out using the moulded microneedles. Individual microneedles were obtained by breaking them from the moulded microneedle strips. The material used was polyetherimide (PEI), an ultra-performance polymer. These individual microneedles (with height of 3mm so that penetration can be easily confirmed by blood) were loaded into a spring-operated applicator which was able to provide a penetration speed of approximately 1 m/sec. 10 consecutive penetrations were performed at a human subjects' fingers and palms. All penetrations were confirmed by visual observations (occurrence of tiny blood spots) and all microneedles were confirmed intact by optical inspection under 200× magnification.

1. A plastic microneedle strip, comprising
a. A thin structure having a base surface;
b. A plurality of microneedles having tapered shapes with one facet substantially flat;
c. Wherein the plurality of microneedles arise from the base surface.

2. The plastic microneedle strip in claim 1, wherein the thin structure has an at least one end with abruptly reduced cross-sectional area for easy separation.

3. The plastic microneedle strip in claim 1, wherein the plurality of microneedle arising from the base surface form one line parallel to the thin structure.

4. A cluster of plastic microneedle strips, comprising
a. A stem for connecting to the plastic microneedle strips;
b. A plurality of plastic microneedle strips having one end with abruptly reduced cross-sectional area for easy separation;
c. Wherein the plurality of plastic microneedle strips are connected to the stem via the ends with abruptly reduced cross-sectional area.

5. A method of making a set of mould inserts for use in injection moulding the plastic microneedle strips in claim 1, comprising
a. Making a first mould insert with a substantially smooth surface;
b. Making a second mould insert with a surface engraved with a plurality of microneedle patterns;
c. Coupling the first and second insert with the smooth surface of the first insert and the engraved surface of the second insert attached to each other firmly to form a complete mould for the plastic microneedle strips.
